(19) United States
(12) Patent Application Publication
(10) Pub. No.: US 2023/0310472 A1
(43) Pub. Date: Oct. 5, 2023

(54) Stable Topical Formulations of 1(R)-4-(5-(4-Methoxy-3-Propoxphenyl)Pyridin-3-YL)-1,2-OX-Aborolan-2-OL

(71) Applicant: Pfizer Inc., New York, NY (US)

(72) Inventor: Thean Yeow Yeoh, Salem, CT (US)

(73) Assignee: Pfizer Inc., New York, NY (US)

(21) Appl. No.: 18/042,054

(22) PCT Filed: Aug. 16, 2021
(86) PCT No.: PCT/IB2021/057518

§ 371 Date: Feb. 17, 2023

Related U.S. Application Data
(60) Provisional application No. 63/222,126, filed on Jul. 15, 2021.
(60) Provisional application No. 63/068,126, filed on Aug. 20, 2020.

(51) Int. Cl. A61K 31/69; A61K 47/10; A61K 47/06; A61K 47/14; A61K 9/00

(52) U.S. Cl. CPC A61K 31/69; A61K 47/10; A61K 47/06; A61K 47/14; A61K 9/0014

(57) ABSTRACT

The present invention relates to the discovery of chemically and physically stable topical formulations comprising (R)-4-(5-(4-methoxy-3-propoxyphenyl)pyridin-3-yl)-1,2-oxaborolan-2-ol (PF-07038124) for treating inflammatory disorders and to methods of preparing the topical formulations.

### FIELD OF THE INVENTION

The present invention relates to the discovery of chemically and physically stable topical formulations comprising (R)-4-(5-(4-methoxy-3-propoxyphenyl)pyridin-3-yl)-1,2-oxaborolan-2-ol (PF-07038124) for treating inflammatory disorders and to methods of preparing the topical formulations.

### BACKGROUND OF THE INVENTION

(R)-4-(5-(4-methoxy-3-propoxyphenyl)pyridin-3-yl)-1,2-oxaborolan-2-ol is a selective and potent phosphodiesterase-4 (PDE4) inhibitor being investigated for the treatment of inflammatory disorders including atopic dermatitis (AD) and psoriasis. Phosphodiesterases (PDEs) represent a family of enzymes that catalyze the hydrolysis of various cyclic nucleoside monophosphates including cyclic adenosine monophosphate (cAMP) and cyclic guanosine monophosphate (cGMP). PDEs regulate the level of cyclic nucleotides within cells and maintain cyclic nucleotide homeostasis by hydrolyzing such cyclic mononucleotides resulting in termination of their messenger role. PDE enzymes can be grouped into families according to their specificity toward hydrolysis of cAMP and/or cGMP, their sensitivity to regulation by calcium and calmodulin, and their selective inhibition by various compounds. The PDE4 enzyme sub-family consists of four genes which produce 4 isoforms of the PDE4 enzyme designated PDE4A, PDE4B, PDE4C, and PDE4D [Wang et al., Biochem. Biophys. Res. Comm., 234, 320 (1997)]. In addition, various splice variants of each PDE4 isoform have been identified. PDE4 isoenzymes specifically inactivate cAMP by catalyzing its hydrolysis to adenosine 5′-monophosphate (AMP). Regulation of cAMP activity is important in many biological processes including inflammation.

AD is an inflammatory skin disease that, typically, manifests during early childhood but can appear in adolescence or adulthood and follows either a chronic or a relapsing/remitting disease progression. AD patients display pruritic skin and show susceptibility to cutaneous secondary bacterial, viral and fungal infections. Patients with AD can also demonstrate a compromised barrier function that leads to activation of keratinocytes and other immune cells. A number of inflammatory cytokines are involved in the symptoms characteristic of AD including, but not limited to, IL-1 IL-2, IL-3, IL-4, IL-5, IL-6, IL-12, IL-13, IL-17, IL-18, IL-22, IL-23, IL-31, IL-33, IL-36, and TNF-α. Inflammatory cytokines facilitate the production of various chemoattractants or chemokines which support the recruitment of leukocytes to the disease site. Chemokines that contribute to inflammation in AD patients include, but not limited to, CCL1, CCL2, CCL3, CCL4, CCLS, CCL11, CCL13, CCL17, CCL18, CCL20, CCL22, CCL26 and CCL27.

There are limited therapeutic options for the treatment of AD. The topical use of anti-inflammatory steroids has been utilized in AD treatment particularly in the case of acute disease flares. The steroids suppress the activation and proliferation of inflammatory cells as well as keratinocytes and fibroblasts. However, steroids can cause adverse local side effects that include, but are not restricted to, skin atrophy, telangiectasia (abnormal dilation of capillary vessels), epidermal barrier disturbance, striae, rosacea, acne, hypertrichosis, hypopigmentation, delayed wound healing and alterations in skin elasticity. Emollients including petrolatum and over-the-counter moisturizers have been used to reduce the use of topical steroids. Topical application of mevalonic acid and nicotinamide has been used to improve the epidermal barrier permeability through the production of cholesterol and ceramide. Topical calcineurin inhibitors (TCI) such as tacrolimus and pimecrolimus have been used in the treatment of AD. Cyclosporine A (CyA) has been used as an immunosuppressant to inhibit calcineurin phosphatase thereby leading to reduction in levels of IL-2 and inhibition of T cell proliferation. Systemic treatment include humanized monoclonal antibodies such as Omalizumab, Efalizumab and Etanercept, Dupilumab that target serum IgE, LFA-1, TNF-α, and IL-4r respectively [Rahman, Inf. & All. 2011, 10, 486]. Additional eczema, skin and disease conditions include hand dermatitis, contact dermatitis, allergic contact dermatitis, irritant contact dermatitis, neurodermatitis, perioral dermatitis, stasis dermatitis, dyshidrotic eczema, xerotic dermatitis, nummalar dermatitis, seborrheic dermatitis, eyelid dermatitis, diaper dermatitis, dermatomyositis, lichen planus, lichen sclerosis, alopecia areata, vitiligo, rosacea, epidermolysis bullosa, keratosis pilaris, pityriasis alba, pemphigus, vulvovaginitis, acne, chronic spontaneous urticaria, chronic idiopathic urticaria, chronic physical urticaria, vogt-koyanagi-harada disease, sutton nevus/nevi, post inflammatory hypopigmentation, senile leukoderma, chemical/drug-induced leukoderma, cutaneous lupus erythematosus, discoid lupus, palmoplantar pustulosis, pemphigoid, sweet's syndrome, and hidradenitis suppurativa [Eyerich and Eyerich, J. Eur. Ac. Derm. Ven., 32, 692 (2018)].

Psoriasis is an immune-mediated chronic skin disease that exists in several different forms including plaque psoriasis, pustular psoriasis, nail psoriasis, flexural psoriasis, guttate psoriasis, psoriatic arthritis, erythrodermic psoriasis, and inverse psoriasis. Plaque psoriasis (psoriasis vulgaris) is the most common form of psoriasis and typically appears as patches of raised red skin covered by a flaky white buildup. Pustular psoriasis appears as raised bumps that are filled with non-infectious pus (pustules). The skin under and surrounding pustules is red and tender. Pustular psoriasis can be localized, commonly to the hands and feet, or generalized with widespread patches occurring on any part of the body. Nail psoriasis produces a variety of changes in the appearance of finger and toe nails. These changes include discoloring under the nail plate, pitting of the nails, lines going across the nails, thickening of the skin under the nail, and the loosening (onycholysis) and crumbling of the nail. Flexural psoriasis (inverse psoriasis) appears as smooth inflamed patches of skin. It occurs in skin folds, particularly around the genitals (between the thigh and groin), the armpits, under an overweight stomach (pannus), and under the breasts (inframammary fold). It is aggravated by friction and sweat and is vulnerable to fungal infections. Guttate psoriasis is characterized by numerous small oval spots. These spots of psoriasis appear over large areas of the body, such as the trunk, limbs, and scalp. Psoriatic arthritis involves joint and connective tissue inflammation. Psoriatic arthritis can affect any joint but is most common in the joints of the fingers and toes. Psoriatic arthritis can result in swelling of the fingers and toes known as dactylitis. Psoriatic arthritis can also affect the hips, knees and spine (spondylitis). Erythrodermic psoriasis involves the widespread inflammation and exfoliation of the skin over most of the body surface. It may be accompanied by severe itching, swelling and pain. It is often the result of an exacerbation of unstable plaque psoriasis, particularly following the abrupt withdrawal of systemic treatment. Current therapies available for treatment of psoriasis include topical treatment, phototherapy, and systemic applications. The treatments are either cosmetically undesirable, inconvenient for long-term use, or have limited effectiveness.

Formulations comprising (R)-4-(5-(4-methoxy-3-propoxyphenyl)pyridin-3-yl)-1,2-oxaborolan-2-ol in recently completed Phase I studies were found to be chemically and physically unstable. Therefore, a need existed for the discovery of topical formulations comprising (R)-4-(5-(4-methoxy-3-propoxyphenyl)pyridin-3-yl)-1,2-oxaborolan-2-ol with improved chemical and physical stability for treating inflammatory disorders such as AD and psoriasis.

### SUMMARY OF THE INVENTION

The present invention provides topical formulations comprising (R)-4-(5-(4-methoxy-3-propoxyphenyl)pyridin-3-yl)-1,2-oxaborolan-2-ol or a pharmaceutically acceptable salt thereof, hexylene glycol, white petrolatum, mono- and di-glycerides, and paraffin wax, wherein the topical formulations are essentially free of butylated hydroxytoluene (BHT) and wherein the topical formulations have improved chemical and physical stability.

The present invention provides a method of treating an inflammatory disorder in a subject comprising topically administering to the subject in need of such treatment a therapeutically effective amount of a topical formulation comprising (R)-4-(5-(4-methoxy-3-propoxyphenyl)pyridin-3-yl)-1,2-oxaborolan-2-ol wherein the topical formulation is prepared from the formulations of the present invention.

The present invention provides methods of preparing topical formulations of the present invention.

### BRIEF DESCRIPTION OF THE FIGURES

FIG. 1A provides comparative chemical stability data for 0.01, 0.03 and 0.1% PF-07038124 formulations comprising 9% HG and 0% BHT at 1, 3 and 6 months.

FIG. 1B provides comparative chemical stability data (% of degradant PF-07086920) for 0.01, 0.03 and 0.06% PF-07038124 formulations comprising 9% HG and 0.1% BHT at 1, 3 and 6 months.

FIG. 2A provides comparative chemical stability data (% of degradant PF-07086920) for 0.01, 0.03 and 0.06% PF-07038124 formulations comprising 5% HG and 0% BHT at 1, 3 and 6 months.

FIG. 2B provides comparative chemical stability data (% of degradant PF-07086920) for 0.01 and 0.03% PF-07038124 formulations comprising 5% HG and 0.1% BHT at 1, 3 and 6 months.

FIG. 3A provides comparative chemical stability data (% of degradant PF-07086920) for 0.01, 0.03 and 0.1% PF-07038124 formulations comprising 2% HG and 0% BHT at 1, 3 and 6 months.

FIG. 3B provides comparative chemical stability data (% of degradant PF-07086920) for 0.01, 0.03 and 0.1% PF-07038124 formulations comprising 2% HG and 0.1% BHT at 1, 3 and 6 months.

FIG. 4 provides comparative chemical stability data for PF-07038124 in formulations comprising: 9% HG and 0% BHT; 5% HG and 0% BHT; and 2% HG and 0% BHT.

FIG. 5 provides a schematic representation of the kilogram scale preparation of topical formulations of the present invention comprising PF-07038124.

FIG. 6 provides a picture of pooling, phase separation of the Phase I formulation (comprising 9% HG and 0.1% BHT) subsequent to opening the bottom valve of the jacketed holding tank and collecting approximately 1 kilogram of the emission in a bucket following overnight static resting in the jacketed holding tank at 29° C. to 35° C.

FIG. 7 provides a picture of no pooling, no phase separation of Formulation L (comprising 5% HG and essentially free of BHT) subsequent to opening the bottom valve of the jacketed holding tank and collecting approximately 1 kilogram of the emission in a bucket following overnight static resting in the jacketed holding tank at 29° C. to 35° C.

### DETAILED DESCRIPTION OF THE INVENTION

According to a first aspect of the invention, there is provided a topical formulation comprising 0.0025% to 2% (R)-4-(5-(4-methoxy-3-propoxyphenyl)pyridin-3-yl)-1,2-oxaborolan-2-ol or a pharmaceutically acceptable salt thereof, 2% to 9% hexylene glycol, quantum satis (qs) white petrolatum, 5% to 9% mono- and di-glycerides, and 3% to 7% paraffin wax, wherein the topical formulation is essentially free of butylated hydroxytoluene and wherein the topical formulation has 3.0% w/w or less degradant diol at 0 to 6 months at 5° C. to 40° C.

Described below are a number of embodiments (E) of this first aspect of the invention, where for convenience E1 is identical thereto.

E1. The topical formulation, according to the first aspect of the invention, as set out just above.

E2. The topical formulation according to embodiment E1, comprising 5% hexylene glycol, 7% mono- and di-glycerides, and 5% paraffin wax.

E3. The topical formulation according to any one of embodiments El to E2, comprising 0.005% to 1.0% (R)-4-(5-(4-methoxy-3-propoxyphenyl)pyridin-3-yl)-1,2-oxaborolan-2-ol or a pharmaceutically acceptable salt thereof.

E4. The topical formulation according to any one of embodiments E1 to E2, comprising 0.005% to 0.5% (R)-4-(5-(4-methoxy-3-propoxyphenyl)pyridin-3-yl)-1,2-oxaborolan-2-ol or a pharmaceutically acceptable salt thereof.

E5. The topical formulation according to any one of embodiments E1 to E2, comprising 0.005% to 0.4% (R)-4-(5-(4-methoxy-3-propoxyphenyl)pyridin-3-yl)-1,2-oxaborolan-2-ol or a pharmaceutically acceptable salt thereof.

E6. The topical formulation according to any one of embodiments E1 to E2, comprising 0.005% to 0.3% (R)-4-(5-(4-methoxy-3-propoxyphenyl)pyridin-3-yl)-1,2-oxaborolan-2-ol or a pharmaceutically acceptable salt thereof.

E7. The topical formulation according to any one of embodiments E1 to E2, comprising 0.005% to 0.2% (R)-4-(5-(4-methoxy-3-propoxyphenyl)pyridin-3-yl)-1,2-oxaborolan-2-ol or a pharmaceutically acceptable salt thereof.

E8. The topical formulation according to any one of embodiments E1 to E2, comprising 0.005% to 0.1% (R)-4-(5-(4-methoxy-3-propoxyphenyl)pyridin-3-yl)-1,2-oxaborolan-2-ol or a pharmaceutically acceptable salt thereof.

E9. The topical formulation according to any one of embodiments E1 to E2, comprising 0.005% to 0.05% (R)-4-(5-(4-methoxy-3-propoxyphenyl)pyridin-3-yl)-1,2-oxaborolan-2-ol or a pharmaceutically acceptable salt thereof.

E10. The topical formulation according to any one of embodiments E1 to E2, comprising 0.005% (R)-4-(5-(4-methoxy-3-propoxyphenyl)pyridin-3-yl)-1,2-oxaborolan-2-ol or a pharmaceutically acceptable salt thereof.

E11. The topical formulation according to any one of embodiments E1 to E2, comprising 0.01% (R)-4-(5-(4-methoxy-3-propoxyphenyl)pyridin-3-yl)-1,2-oxaborolan-2-ol or a pharmaceutically acceptable salt thereof.

E12. The topical formulation according to any one of embodiments E1 to E2, comprising 0.02% (R)-4-(5-(4-methoxy-3-propoxyphenyl)pyridin-3-yl)-1,2-oxaborolan-2-ol or a pharmaceutically acceptable salt thereof.

E13. The topical formulation according to any one of embodiments E1 to E2, comprising 0.03% (R)-4-(5-(4-methoxy-3-propoxyphenyl)pyridin-3-yl)-1,2-oxaborolan-2-ol or a pharmaceutically acceptable salt thereof.

E14. The topical formulation according to any one of embodiments E1 or E2, comprising 0.04% (R)-4-(5-(4-methoxy-3-propoxyphenyl)pyridin-3-yl)-1,2-oxaborolan-2-ol or a pharmaceutically acceptable salt thereof.

E15. The topical formulation according to any one of embodiments E1 or E2, comprising 0.05% (R)-4-(5-(4-methoxy-3-propoxyphenyl)pyridin-3-yl)-1,2-oxaborolan-2-ol or a pharmaceutically acceptable salt thereof.

E16. The topical formulation according to any one of embodiments E1 or E2, comprising 0.06% (R)-4-(5-(4-methoxy-3-propoxyphenyl)pyridin-3-yl)-1,2-oxaborolan-2-ol or a pharmaceutically acceptable salt thereof.

E17. The topical formulation according to any one of embodiments E1 or E2, comprising 0.07% (R)-4-(5-(4-methoxy-3-propoxyphenyl)pyridin-3-yl)-1,2-oxaborolan-2-ol or a pharmaceutically acceptable salt thereof.

E18. The topical formulation according to any one of embodiments E1 or E2, comprising 0.08% (R)-4-(5-(4-methoxy-3-propoxyphenyl)pyridin-3-yl)-1,2-oxaborolan-2-ol or a pharmaceutically acceptable salt thereof.

E19. The topical formulation according to any one of embodiments E1 or E2, comprising 0.09% (R)-4-(5-(4-methoxy-3-propoxyphenyl)pyridin-3-yl)-1,2-oxaborolan-2-ol or a pharmaceutically acceptable salt thereof.

E20. The topical formulation according to any one of embodiments E1 or E2, comprising 0.1% (R)-4-(5-(4-methoxy-3-propoxyphenyl)pyridin-3-yl)-1,2-oxaborolan-2-ol or a pharmaceutically acceptable salt thereof.

E21. The topical formulation according to any one of embodiments E1 or E2, comprising 0.2% (R)-4-(5-(4-methoxy-3-propoxyphenyl)pyridin-3-yl)-1,2-oxaborolan-2-ol or a pharmaceutically acceptable salt thereof.

E22. The topical formulation according to any one of embodiments E1 or E2, comprising 0.3% (R)-4-(5-(4-methoxy-3-propoxyphenyl)pyridin-3-yl)-1,2-oxaborolan-2-ol or a pharmaceutically acceptable salt thereof.

E23. The topical formulation according to any one of embodiments E1 or E2, comprising 0.4% (R)-4-(5-(4-methoxy-3-propoxyphenyl)pyridin-3-yl)-1,2-oxaborolan-2-ol or a pharmaceutically acceptable salt thereof.

E24. The topical formulation according to any one of embodiments E1 or E2, comprising 0.5% (R)-4-(5-(4-methoxy-3-propoxyphenyl)pyridin-3-yl)-1,2-oxaborolan-2-ol or a pharmaceutically acceptable salt thereof.

E25. The topical formulation according to any one of embodiments E1 or E2, comprising 0.6% (R)-4-(5-(4-methoxy-3-propoxyphenyl)pyridin-3-yl)-1,2-oxaborolan-2-ol or a pharmaceutically acceptable salt thereof.

E26. The topical formulation according to any one of embodiments E1 or E2, comprising 0.7% (R)-4-(5-(4-methoxy-3-propoxyphenyl)pyridin-3-yl)-1,2-oxaborolan-2-ol or a pharmaceutically acceptable salt thereof.

E27. The topical formulation according to any one of embodiments E1 or E2, comprising 0.8% (R)-4-(5-(4-methoxy-3-propoxyphenyl)pyridin-3-yl)-1,2-oxaborolan-2-ol or a pharmaceutically acceptable salt thereof.

E28. The topical formulation according to any one of embodiments E1 or E2, comprising 0.9% (R)-4-(5-(4-methoxy-3-propoxyphenyl)pyridin-3-yl)-1,2-oxaborolan-2-ol or a pharmaceutically acceptable salt thereof.

E29. The topical formulation according to any one of embodiments E1 or E2, comprising 1.0% (R)-4-(5-(4-methoxy-3-propoxyphenyl)pyridin-3-yl)-1,2-oxaborolan-2-ol or a pharmaceutically acceptable salt thereof.

E30. The topical formulation according to any one of embodiments E1 or E2, comprising 1.5% (R)-4-(5-(4-methoxy-3-propoxyphenyl)pyridin-3-yl)-1,2-oxaborolan-2-ol or a pharmaceutically acceptable salt thereof.

E31. The topical formulation according to any one of embodiments E1 or E2, comprising 2.0% (R)-4-(5-(4-methoxy-3-propoxyphenyl)pyridin-3-yl)-1,2-oxaborolan-2-ol or a pharmaceutically acceptable salt thereof.

E32. The topical formulation according to any one of embodiments E1 to E31, wherein the topical formulation has 2.0% w/w or less degradant diol at 0 to 6 months at 5° C. to 40° C.

E33. The topical formulation according to any one of embodiments E1 to E31, wherein the topical formulation has 1.0% w/w or less degradant diol at 0 to 6 months at 5° C. to 40° C.

E34. The topical formulation according to any one of embodiments E1 to E31, wherein the topical formulation has 1.0% w/w or less degradant diol at 6 months at 5° C.

E35. The topical formulation according to any one of embodiments E1 to E31, wherein the topical formulation has 1.0% w/w or less degradant diol at 6 months at 25° C.

E36. The topical formulation according to any one of embodiments E1 to E31, wherein the topical formulation has 1.0% w/w or less degradant diol at 6 months at 40° C.

E37. The topical formulation according to any one of embodiments E1 to E31, wherein the topical formulation has 1.0% w/w or less degradant diol at 0 to 12 months at 5° C. to 25° C.

E38. The topical formulation according to any one of embodiments E1 to E31, wherein the topical formulation has 1.0% w/w or less degradant diol at 12 months at 5° C.

E39. The topical formulation according to any one of embodiments E1 to E31, wherein the topical formulation has 1.0% w/w or less degradant diol at 12 months at 25° C.

E40. A method for treating a disease or condition selected from the group consisting of psoriasis, plaque psoriasis, pustular psoriasis, nail psoriasis, flexural psoriasis, guttate psoriasis, psoriatic arthritis, erythrodermic psoriasis, and inverse psoriasis in a subject comprising administering to the subject in need of such treatment a therapeutically effective amount of the topical formulation according to any one of embodiments E1 to E39.

E41. The method according to embodiment E40, wherein the disease or condition is plaque psoriasis.

E42. The method according to embodiment E40, wherein the disease or condition is psoriasis.

E43. The topical formulation according to any one of embodiments E1 to E39 for use in the treatment of psoriasis, plaque psoriasis, pustular psoriasis, nail psoriasis, flexural psoriasis, guttate psoriasis, psoriatic arthritis, erythrodermic psoriasis, or inverse psoriasis.

E44. The topical formulation according to any one of embodiments E1 to E39 for use in the treatment of plaque psoriasis.

E45. The topical formulation according to any one of embodiments El to E39 for use in the treatment of psoriasis.

E46. Use of the topical formulation according to any one of embodiments E1 to E39 in the manufacture of a medicament for use in the treatment of psoriasis, plaque psoriasis, pustular psoriasis, nail psoriasis, flexural psoriasis, guttate psoriasis, psoriatic arthritis, erythrodermic psoriasis, or inverse psoriasis.

E47. Use of the topical formulation according to any one of embodiments E1 to E39 in the manufacture of a medicament for use in the treatment of plaque psoriasis.

E48. Use of the topical formulation according to any one of embodiments E1 to E39 in the manufacture of a medicament for use in the treatment of psoriasis.

E49. A method for treating a disease or condition selected from the group consisting of eczema, atopic dermatitis, hand dermatitis, contact dermatitis, allergic contact dermatitis, irritant contact dermatitis, neurodermatitis, perioral dermatitis, stasis dermatitis, dyshidrotic eczema, xerotic dermatitis, nummalar dermatitis, seborrheic dermatitis, eyelid dermatitis, diaper dermatitis, dermatomyositis, lichen planus, lichen sclerosis, alopecia areata, vitiligo, rosacea, epidermolysis bullosa, keratosis pilaris, pityriasis alba, pemphigus, vulvovaginitis, acne, chronic spontaneous urticaria, chronic idiopathic urticaria, chronic physical urticaria, vogt-koyanagi-harada disease, sutton nevus/nevi, post inflammatory hypopigmentation, senile leukoderma, chemical/drug-induced leukoderma, cutaneous lupus erythematosus, discoid lupus, palmoplantar pustulosis, pemphigoid, sweet's syndrome, and hidradenitis suppurativa in a subject comprising administering to the subject in need of such treatment a therapeutically effective amount of the topical formulation according to any one of embodiments E1 to E39.

E50. The method according to embodiment E49, wherein the disease or condition is atopic dermatitis.

E51. The method according to embodiment E49, wherein the disease or condition is eczema.

E52. The topical formulation according to any one of embodiments E1 to E39 for use in the treatment of eczema, atopic dermatitis, hand dermatitis, contact dermatitis, allergic contact dermatitis, irritant contact dermatitis, neurodermatitis, perioral dermatitis, stasis dermatitis, dyshidrotic eczema, xerotic dermatitis, nummalar dermatitis, seborrheic dermatitis, eyelid dermatitis, diaper dermatitis, dermatomyositis, lichen planus, lichen sclerosis, alopecia areata, vitiligo, rosacea, epidermolysis bullosa, keratosis pilaris, pityriasis alba, pemphigus, vulvovaginitis, acne, chronic spontaneous urticaria, chronic idiopathic urticaria, chronic physical urticaria, vogt-koyanagi-harada disease, sutton nevus/nevi, post inflammatory hypopigmentation, senile leukoderma, chemical/drug-induced leukoderma, cutaneous lupus erythematosus, discoid lupus, palmoplantar pustulosis, pemphigoid, sweet's syndrome, and hidradenitis suppurativa.

E53. The topical formulation according to any one of embodiments E1 to E39 for use in the treatment of atopic dermatitis.

E54. The topical formulation according to any one of embodiments E1 to E39 for use in the treatment of eczema.

E55. Use of the topical formulation according to any one of embodiments E1 to E39 in the manufacture of a medicament for use in the treatment of eczema, atopic dermatitis, hand dermatitis, contact dermatitis, allergic contact dermatitis, irritant contact dermatitis, neurodermatitis, perioral dermatitis, stasis dermatitis, dyshidrotic eczema, xerotic dermatitis, nummalar dermatitis, seborrheic dermatitis, eyelid dermatitis, diaper dermatitis, dermatomyositis, lichen planus, lichen sclerosis, alopecia areata, vitiligo, rosacea, epidermolysis bullosa, keratosis pilaris, pityriasis alba, pemphigus, vulvovaginitis, acne, chronic spontaneous urticaria, chronic idiopathic urticaria, chronic physical urticaria, vogt-koyanagi-harada disease, sutton nevus/nevi, post inflammatory hypopigmentation, senile leukoderma, chemical/drug-induced leukoderma, cutaneous lupus erythematosus, discoid lupus, palmoplantar pustulosis, pemphigoid, sweet's syndrome, and hidradenitis suppurativa.

E56. Use of the topical formulation according to any one of embodiments E1 to E39 in the manufacture of a medicament for use in the treatment of atopic dermatitis.

E57. Use of the topical formulation according to any one of embodiments E1 to E39 in the manufacture of a medicament for use in the treatment of eczema.

E58. A method of preparing a topical formulation according to any one of embodiments E1 to E39, comprising:

1. mixing (R)-4-(5-(4-methoxy-3-propoxyphenyl)pyridin-3-yl)-1,2-oxaborolan-2-ol, or a pharmaceutically acceptable salt thereof, and hexylene glycol in a vessel to provide an active phase;

2. mixing white petrolatum, mono- and di-glycerides, and paraffin wax in a jacketed vessel to provide a wax phase;

3. transferring the wax phase into a homogenization tank;

4. transferring the active phase into the homogenization tank;

5. homogenizing the wax phase and the active phase in the homogenization tank with a high shear bottom mounted dispersion-homogenizer unit (DISHO); and

6. transferring the topical formulation into a jacketed holding vessel wherein the topical formulation is held static overnight at 29° C. to 35° C. and wherein there is no pooling or phase separation of the topical formulation as determined by unenhanced visual inspection.

E59. The method according to embodiment E58 wherein the jacketed vessel of step 2 is heated to 70° C. to 80° C. with mixing until dissolution of the wax phase.

E60. The method according to embodiments E58 and E59 wherein the homogenization tank of step 3 is heated to 70° C. to 80° C. with recirculation for not less than 10 minutes and then cooled to 40° C. to 46° C. with mixing and recirculation.

E61. The method according to embodiments E58 to E60 wherein the homogenization tank of step 5 is maintained at 40° C. to 46° C. and the wax phase and the active phase homogenized for not less than 60 minutes with the DISHO at 1600 rpm.

E62. The method according to embodiment E61 wherein the DISHO rpm is adjusted to maintain the temperature at 40° C. to 46° C.

E63. The method according to embodiments E61 and E62 wherein after the homogenization of the wax phase and the active phase, the homogenization tank of step 5 is cooled to 29° C. to 35° C. and the DISHO rpm reduced to 1000 rpm.

E64. The method according to embodiment E63 wherein the DISHO rpm is adjusted to maintain, at a minimum, recirculation.

E65. The method according to embodiments E58 to E64 wherein the jacketed holding tank of step 6 is maintained at 29° C. to 35° C. during the transfer of the topical formulation from the homogenization tank to the jacketed holding tank.

E66. A topical formulation comprising 0.0025% to 2% (R)-4-(5-(4-methoxy-3-propoxyphenyl)pyridin-3-yl)-1,2-oxaborolan-2-ol or a pharmaceutically acceptable salt thereof, 2% to 9% hexylene glycol, quantum satis (qs) white petrolatum, 5% to 9% mono- and di-glycerides, and 3% to 7% paraffin wax.

E67. The topical formulation of E66 comprising 3% to 7% hexylene glycol.

E68. The topical formulation of E67 comprising 4% to 6% hexylene glycol.

E69. The topical formulation of E68 comprising 4.5% to 5.5% hexylene glycol.

E70. The topical formulation of E69 comprising 5% hexylene glycol.

E71. The topical formulation of E66 comprising 2% to 5% hexylene glycol.

E72. The topical formulation of any one of E66 to E71 comprising 5% hexylene glycol, 7% mono- and di-glycerides and 5% paraffin wax.

E73. The topical formulation according to any one of embodiments E66 to E72, comprising 0.005% to 1.0% (R)-4-(5-(4-methoxy-3-propoxyphenyl)pyridin-3-yl)-1,2-oxaborolan-2-ol or a pharmaceutically acceptable salt thereof.

E74. The topical formulation according to any one of embodiments E66 to E72, comprising 0.005% to 0.5% (R)-4-(5-(4-methoxy-3-propoxyphenyl)pyridin-3-yl)-1,2-oxaborolan-2-ol or a pharmaceutically acceptable salt thereof.

E75. The topical formulation according to any one of embodiments E66 to E72, comprising 0.005% to 0.4% (R)-4-(5-(4-methoxy-3-propoxyphenyl)pyridin-3-yl)-1,2-oxaborolan-2-ol or a pharmaceutically acceptable salt thereof.

E76. The topical formulation according to any one of embodiments E66 to E72, comprising 0.005% to 0.3% (R)-4-(5-(4-methoxy-3-propoxyphenyl)pyridin-3-yl)-1,2-oxaborolan-2-ol or a pharmaceutically acceptable salt thereof.

E77. The topical formulation according to any one of embodiments E66 to E72, comprising 0.005% to 0.2% (R)-4-(5-(4-methoxy-3-propoxyphenyl)pyridin-3-yl)-1,2-oxaborolan-2-ol or a pharmaceutically acceptable salt thereof.

E78. The topical formulation according to any one of embodiments E66 to E72, comprising 0.005% to 0.1% (R)-4-(5-(4-methoxy-3-propoxyphenyl)pyridin-3-yl)-1,2-oxaborolan-2-ol or a pharmaceutically acceptable salt thereof.

E79. The topical formulation according to any one of embodiments E66 to E72, comprising 0.005% to 0.05% (R)-4-(5-(4-methoxy-3-propoxphenyl)pyridin-3-yl)-1,2-oxaborolan-2-ol or a pharmaceutically acceptable salt thereof.

E80. The topical formulation according to any one of embodiments E66 to E72, comprising 0.005% (R)-4-(5-(4-methoxy-3-propoxyphenyl)pyridin-3-yl)-1,2-oxaborolan-2-ol or a pharmaceutically acceptable salt thereof.

E81. The topical formulation according to any one of embodiments E66 to E72, comprising 0.01% (R)-4-(5-(4-methoxy-3-propoxyphenyl)pyridin-3-yl)-1,2-oxaborolan-2-ol or a pharmaceutically acceptable salt thereof.

E82. The topical formulation according to any one of embodiments E66 to E72, comprising 0.02% (R)-4-(5-(4-methoxy-3-propoxyphenyl)pyridin-3-yl)-1,2-oxaborolan-2-ol or a pharmaceutically acceptable salt thereof.

E83. The topical formulation according to any one of embodiments E66 to E72, comprising 0.03% (R)-4-(5-(4-methoxy-3-propoxyphenyl)pyridin-3-yl)-1,2-oxaborolan-2-ol or a pharmaceutically acceptable salt thereof.

E84. The topical formulation according to any one of embodiments E66 to E72, comprising 0.04% (R)-4-(5-(4-methoxy-3-propoxyphenyl)pyridin-3-yl)-1,2-oxaborolan-2-ol or a pharmaceutically acceptable salt thereof.

E85. The topical formulation according to any one of embodiments E66 to E72, comprising 0.05% (R)-4-(5-(4-methoxy-3-propoxyphenyl)pyridin-3-yl)-1,2-oxaborolan-2-ol or a pharmaceutically acceptable salt thereof.

E86. The topical formulation according to any one of embodiments E66 to E72, comprising 0.06% (R)-4-(5-(4-methoxy-3-propoxyphenyl)pyridin-3-yl)-1,2-oxaborolan-2-ol or a pharmaceutically acceptable salt thereof.

E87. The topical formulation according to any one of embodiments E66 to E72, comprising 0.07% (R)-4-(5-(4-methoxy-3-propoxyphenyl)pyridin-3-yl)-1,2-oxaborolan-2-ol or a pharmaceutically acceptable salt thereof.

E88. The topical formulation according to any one of embodiments E66 to E72, comprising 0.08% (R)-4-(5-(4-methoxy-3-propoxyphenyl)pyridin-3-yl)-1,2-oxaborolan-2-ol or a pharmaceutically acceptable salt thereof.

E89. The topical formulation according to any one of embodiments E66 to E72, comprising 0.09% (R)-4-(5-(4-methoxy-3-propoxyphenyl)pyridin-3-yl)-1,2-oxaborolan-2-ol or a pharmaceutically acceptable salt thereof.

E90. The topical formulation according to any one of embodiments E66 to E72, comprising 0.1% (R)-4-(5-(4-methoxy-3-propoxyphenyl)pyridin-3-yl)-1,2-oxaborolan-2-ol or a pharmaceutically acceptable salt thereof.

E91. The topical formulation according to any one of embodiments E66 to E72, comprising 0.2% (R)-4-(5-(4-methoxy-3-propoxyphenyl)pyridin-3-yl)-1,2-oxaborolan-2-ol or a pharmaceutically acceptable salt thereof.

E92. The topical formulation according to any one of embodiments E66 to E72, comprising 0.3% (R)-4-(5-(4-methoxy-3-propoxyphenyl)pyridin-3-yl)-1,2-oxaborolan-2-ol or a pharmaceutically acceptable salt thereof.

E93. The topical formulation according to any one of embodiments E66 to E72, comprising 0.4% (R)-4-(5-(4-methoxy-3-propoxyphenyl)pyridin-3-yl)-1,2-oxaborolan-2-ol or a pharmaceutically acceptable salt thereof.

E94. The topical formulation according to any one of embodiments E66 to E72, comprising 0.5% (R)-4-(5-(4-methoxy-3-propoxyphenyl)pyridin-3-yl)-1,2-oxaborolan-2-ol or a pharmaceutically acceptable salt thereof.

E95. The topical formulation according to any one of embodiments E66 to E72, comprising 0.6% (R)-4-(5-(4-methoxy-3-propoxyphenyl)pyridin-3-yl)-1,2-oxaborolan-2-ol or a pharmaceutically acceptable salt thereof.

E96. The topical formulation according to any one of embodiments E66 to E72, comprising 0.7% (R)-4-(5-(4-methoxy-3-propoxyphenyl)pyridin-3-yl)-1,2-oxaborolan-2-ol or a pharmaceutically acceptable salt thereof.

E97. The topical formulation according to any one of embodiments E66 to E72, comprising 0.8% (R)-4-(5-(4-methoxy-3-propoxyphenyl)pyridin-3-yl)-1,2-oxaborolan-2-ol or a pharmaceutically acceptable salt thereof.

E98. The topical formulation according to any one of embodiments E66 to E72, comprising 0.9% (R)-4-(5-(4-methoxy-3-propoxyphenyl)pyridin-3-yl)-1,2-oxaborolan-2-ol or a pharmaceutically acceptable salt thereof.

E99. The topical formulation according to any one of embodiments E66 to E2, comprising 1.0% (R)-4-(5-(4-methoxy-3-propoxyphenyl)pyridin-3-yl)-1,2-oxaborolan-2-ol or a pharmaceutically acceptable salt thereof.

E100. The topical formulation according to any one of embodiments E66 to E72, comprising 1.5% (R)-4-(5-(4-methoxy-3-propoxyphenyl)pyridin-3-yl)-1,2-oxaborolan-2-ol or a pharmaceutically acceptable salt thereof.

E101. The topical formulation according to any one of embodiments E66 to E72, comprising 2.0% (R)-4-(5-(4-methoxy-3-propoxyphenyl)pyridin-3-yl)-1,2-oxaborolan-2-ol or a pharmaceutically acceptable salt thereof.

E102. A topical formulation comprising 0.0025% to 2% (R)-4-(5-(4-methoxy-3-propoxyphenyl)pyridin-3-yl)-1,2-oxaborolan-2-ol or a pharmaceutically acceptable salt thereof, 2% to 9% hexylene glycol, quantum satis (qs) white petrolatum, 4% to 9% triglycerides, and 3% to 7% paraffin wax.

E103. The topical formulation of E102 comprising 3% to 7% hexylene glycol.

E104. The topical formulation of E103 comprising 4% to 6% hexylene glycol.

E105. The topical formulation of E104 comprising 4.5% to 5.5% hexylene glycol.

E106. The topical formulation of E105 comprising 5% hexylene glycol.

E107. The topical formulation of E102 comprising 2% to 5% hexylene glycol.

E108. The topical formulation of any one of E102 to E107 comprising 5% hexylene glycol, 5 to 8% triglycerides and 5% paraffin wax.

E109. The topical formulation according to any one of embodiments E102 to E108, comprising 0.005% to 1.0% (R)-4-(5-(4-methoxy-3-propoxyphenyl)pyridin-3-yl)-1,2-oxaborolan-2-ol or a pharmaceutically acceptable salt thereof.

E110. The topical formulation according to any one of embodiments E102 to E108, comprising 0.005% to 0.5% (R)-4-(5-(4-methoxy-3-propoxyphenyl)pyridin-3-yl)-1,2-oxaborolan-2-ol or a pharmaceutically acceptable salt thereof.

E111. The topical formulation according to any one of embodiments E102 to E108, comprising 0.005% to 0.4% (R)-4-(5-(4-methoxy-3-propoxyphenyl)pyridin-3-yl)-1,2-oxaborolan-2-ol or a pharmaceutically acceptable salt thereof.

E112. The topical formulation according to any one of embodiments E102 to E108, comprising 0.005% to 0.3% (R)-4-(5-(4-methoxy-3-propoxyphenyl)pyridin-3-yl)-1,2-oxaborolan-2-ol or a pharmaceutically acceptable salt thereof.

E113. The topical formulation according to any one of embodiments E102 to E108, comprising 0.005% to 0.2% (R)-4-(5-(4-methoxy-3-propoxyphenyl)pyridin-3-yl)-1,2-oxaborolan-2-ol or a pharmaceutically acceptable salt thereof.

E114. The topical formulation according to any one of embodiments E102 to E108, comprising 0.005% to 0.1% (R)-4-(5-(4-methoxy-3-propoxyphenyl)pyridin-3-yl)-1,2-oxaborolan-2-ol or a pharmaceutically acceptable salt thereof.

E115. The topical formulation according to any one of embodiments E102 to E108, comprising 0.005% to 0.05% (R)-4-(5-(4-methoxy-3-propoxphenyl)pyridin-3-yl)-1,2-oxaborolan-2-ol or a pharmaceutically acceptable salt thereof.

E116. The topical formulation according to any one of embodiments E102 to E108, comprising 0.005% (R)-4-(5-(4-methoxy-3-propoxyphenyl)pyridin-3-yl)-1,2-oxaborolan-2-ol or a pharmaceutically acceptable salt thereof.

E117. The topical formulation according to any one of embodiments E102 to E108, comprising 0.01% (R)-4-(5-(4-methoxy-3-propoxyphenyl)pyridin-3-yl)-1,2-oxaborolan-2-ol or a pharmaceutically acceptable salt thereof.

E118. The topical formulation according to any one of embodiments E102 to E108, comprising 0.02% (R)-4-(5-(4-methoxy-3-propoxyphenyl)pyridin-3-yl)-1,2-oxaborolan-2-ol or a pharmaceutically acceptable salt thereof.

E119. The topical formulation according to any one of embodiments E102 to E108, comprising 0.03% (R)-4-(5-(4-methoxy-3-propoxyphenyl)pyridin-3-yl)-1,2-oxaborolan-2-ol or a pharmaceutically acceptable salt thereof.

E120. The topical formulation according to any one of embodiments E102 to E108, comprising 0.04% (R)-4-(5-(4-methoxy-3-propoxyphenyl)pyridin-3-yl)-1,2-oxaborolan-2-ol or a pharmaceutically acceptable salt thereof.

E121. The topical formulation according to any one of embodiments E102 to E108, comprising 0.05% (R)-4-(5-(4-methoxy-3-propoxyphenyl)pyridin-3-yl)-1,2-oxaborolan-2-ol or a pharmaceutically acceptable salt thereof.

E122. The topical formulation according to any one of embodiments E102 to E108, comprising 0.06% (R)-4-(5-(4-methoxy-3-propoxyphenyl)pyridin-3-yl)-1,2-oxaborolan-2-ol or a pharmaceutically acceptable salt thereof.

E123. The topical formulation according to any one of embodiments E102 to E108, comprising 0.07% (R)-4-(5-(4-methoxy-3-propoxyphenyl)pyridin-3-yl)-1,2-oxaborolan-2-ol or a pharmaceutically acceptable salt thereof.

E124. The topical formulation according to any one of embodiments E102 to E108, comprising 0.08% (R)-4-(5-(4-methoxy-3-propoxyphenyl)pyridin-3-yl)-1,2-oxaborolan-2-ol or a pharmaceutically acceptable salt thereof.

E125. The topical formulation according to any one of embodiments E102 to E108, comprising 0.09% (R)-4-(5-(4-methoxy-3-propoxyphenyl)pyridin-3-yl)-1,2-oxaborolan-2-ol or a pharmaceutically acceptable salt thereof.

E126. The topical formulation according to any one of embodiments E102 to E108, comprising 0.1% (R)-4-(5-(4-methoxy-3-propoxyphenyl)pyridin-3-yl)-1,2-oxaborolan-2-ol or a pharmaceutically acceptable salt thereof.

E127. The topical formulation according to any one of embodiments E102 to E108, comprising 0.2% (R)-4-(5-(4-methoxy-3-propoxyphenyl)pyridin-3-yl)-1,2-oxaborolan-2-ol or a pharmaceutically acceptable salt thereof.

E128. The topical formulation according to any one of embodiments E102 to E108, comprising 0.3% (R)-4-(5-(4-methoxy-3-propoxyphenyl)pyridin-3-yl)-1,2-oxaborolan-2-ol or a pharmaceutically acceptable salt thereof.

E129. The topical formulation according to any one of embodiments E102 to E108, comprising 0.4% (R)-4-(5-(4-methoxy-3-propoxyphenyl)pyridin-3-yl)-1,2-oxaborolan-2-ol or a pharmaceutically acceptable salt thereof.

E130. The topical formulation according to any one of embodiments E102 to E108, comprising 0.5% (R)-4-(5-(4-methoxy-3-propoxyphenyl)pyridin-3-yl)-1,2-oxaborolan-2-ol or a pharmaceutically acceptable salt thereof.

E131. The topical formulation according to any one of embodiments E102 to E108, comprising 0.6% (R)-4-(5-(4-methoxy-3-propoxyphenyl)pyridin-3-yl)-1,2-oxaborolan-2-ol or a pharmaceutically acceptable salt thereof.

E132. The topical formulation according to any one of embodiments E102 to E108, comprising 0.7% (R)-4-(5-(4-methoxy-3-propoxyphenyl)pyridin-3-yl)-1,2-oxaborolan-2-ol or a pharmaceutically acceptable salt thereof.

E133. The topical formulation according to any one of embodiments E102 to E108, comprising 0.8% (R)-4-(5-(4-methoxy-3-propoxyphenyl)pyridin-3-yl)-1,2-oxaborolan-2-ol or a pharmaceutically acceptable salt thereof.

E134. The topical formulation according to any one of embodiments E102 to E108, comprising 0.9% (R)-4-(5-(4-methoxy-3-propoxyphenyl)pyridin-3-yl)-1,2-oxaborolan-2-ol or a pharmaceutically acceptable salt thereof.

E135. The topical formulation according to any one of embodiments E102 to E108, comprising 1.0% (R)-4-(5-(4-methoxy-3-propoxyphenyl)pyridin-3-yl)-1,2-oxaborolan-2-ol or a pharmaceutically acceptable salt thereof.

E136. The topical formulation according to any one of embodiments E102 to E108, comprising 1.5% (R)-4-(5-(4-methoxy-3-propoxyphenyl)pyridin-3-yl)-1,2-oxaborolan-2-ol or a pharmaceutically acceptable salt thereof.

E137. The topical formulation according to any one of embodiments E102 to E108, comprising 2.0% (R)-4-(5-(4-methoxy-3-propoxyphenyl)pyridin-3-yl)-1,2-oxaborolan-2-ol or a pharmaceutically acceptable salt thereof.

E138. A topical formulation comprising 0.0025% to 2% (R)-4-(5-(4-methoxy-3-propoxyphenyl)pyridin-3-yl)-1,2-oxaborolan-2-ol or a pharmaceutically acceptable salt thereof, 2% to 9% hexylene glycol, quantum satis (qs) white petrolatum, 4% to 9% mono-, di- and triglycerides, and 3% to 7% paraffin wax.

E139. The topical formulation of E138 comprising 3% to 7% hexylene glycol.

E140. The topical formulation of E139 comprising 4% to 6% hexylene glycol.

E141. The topical formulation of E140 comprising 4.5% to 5.5% hexylene glycol.

E142. The topical formulation of E141 comprising 5% hexylene glycol.

E143. The topical formulation of E138 comprising 2% to 5% hexylene glycol.

E144. The topical formulation of any one of E138 to E143 comprising 5% hexylene glycol, 5 to 8% mono-, di- and triglycerides and 5% paraffin wax.

E145. The topical formulation of any one of E138 to E144 wherein the mono-, di- and triglycerides comprises 12 to 18% (w/w) glycerol monobehenate, 45 to 54% (w/w) glycerol dibehenate and 28 to 32% of glycerol tribehenate.

E146. The topical formulation according to any one of embodiments E138 to E145, comprising 0.005% to 1.0% (R)-4-(5-(4-methoxy-3-propoxyphenyl)pyridin-3-yl)-1,2-oxaborolan-2-ol or a pharmaceutically acceptable salt thereof.

E147. The topical formulation according to any one of embodiments E138 to E145, comprising 0.005% to 0.5% (R)-4-(5-(4-methoxy-3-propoxyphenyl)pyridin-3-yl)-1,2-oxaborolan-2-ol or a pharmaceutically acceptable salt thereof.

E148. The topical formulation according to any one of embodiments E138 to E145, comprising 0.005% to 0.4% (R)-4-(5-(4-methoxy-3-propoxyphenyl)pyridin-3-yl)-1,2-oxaborolan-2-ol or a pharmaceutically acceptable salt thereof.

E149. The topical formulation according to any one of embodiments E138 to E145, comprising 0.005% to 0.3% (R)-4-(5-(4-methoxy-3-propoxyphenyl)pyridin-3-yl)-1,2-oxaborolan-2-ol or a pharmaceutically acceptable salt thereof.

E150. The topical formulation according to any one of embodiments E138 to E145, comprising 0.005% to 0.2% (R)-4-(5-(4-methoxy-3-propoxyphenyl)pyridin-3-yl)-1,2-oxaborolan-2-ol or a pharmaceutically acceptable salt thereof.

E151. The topical formulation according to any one of embodiments E138 to E145, comprising 0.005% to 0.1% (R)-4-(5-(4-methoxy-3-propoxyphenyl)pyridin-3-yl)-1,2-oxaborolan-2-ol or a pharmaceutically acceptable salt thereof.

E152. The topical formulation according to any one of embodiments E138 to E145, comprising 0.005% to 0.05% (R)-4-(5-(4-methoxy-3-propoxphenyl)pyridin-3-yl)-1,2-oxaborolan-2-ol or a pharmaceutically acceptable salt thereof.

E153. The topical formulation according to any one of embodiments E138 to E145, comprising 0.005% (R)-4-(5-(4-methoxy-3-propoxyphenyl)pyridin-3-yl)-1,2-oxaborolan-2-ol or a pharmaceutically acceptable salt thereof.

E154. The topical formulation according to any one of embodiments E138 to E145, comprising 0.01% (R)-4-(5-(4-methoxy-3-propoxyphenyl)pyridin-3-yl)-1,2-oxaborolan-2-ol or a pharmaceutically acceptable salt thereof.

E155. The topical formulation according to any one of embodiments E138 to E145, comprising 0.02% (R)-4-(5-(4-methoxy-3-propoxyphenyl)pyridin-3-yl)-1,2-oxaborolan-2-ol or a pharmaceutically acceptable salt thereof.

E156. The topical formulation according to any one of embodiments E138 to E145, comprising 0.03% (R)-4-(5-(4-methoxy-3-propoxyphenyl)pyridin-3-yl)-1,2-oxaborolan-2-ol or a pharmaceutically acceptable salt thereof.

E157. The topical formulation according to any one of embodiments E138 to E145, comprising 0.04% (R)-4-(5-(4-methoxy-3-propoxyphenyl)pyridin-3-yl)-1,2-oxaborolan-2-ol or a pharmaceutically acceptable salt thereof.

E158. The topical formulation according to any one of embodiments E138 to E145, comprising 0.05% (R)-4-(5-(4-methoxy-3-propoxyphenyl)pyridin-3-yl)-1,2-oxaborolan-2-ol or a pharmaceutically acceptable salt thereof.

E159. The topical formulation according to any one of embodiments E138 to E145, comprising 0.06% (R)-4-(5-(4-methoxy-3-propoxyphenyl)pyridin-3-yl)-1,2-oxaborolan-2-ol or a pharmaceutically acceptable salt thereof.

E160. The topical formulation according to any one of embodiments E138 to E145, comprising 0.07% (R)-4-(5-(4-methoxy-3-propoxyphenyl)pyridin-3-yl)-1,2-oxaborolan-2-ol or a pharmaceutically acceptable salt thereof.

E161. The topical formulation according to any one of embodiments E138 to E145, comprising 0.08% (R)-4-(5-(4-methoxy-3-propoxyphenyl)pyridin-3-yl)-1,2-oxaborolan-2-ol or a pharmaceutically acceptable salt thereof.

E162. The topical formulation according to any one of embodiments E138 to E145, comprising 0.09% (R)-4-(5-(4-methoxy-3-propoxyphenyl)pyridin-3-yl)-1,2-oxaborolan-2-ol or a pharmaceutically acceptable salt thereof.

E163. The topical formulation according to any one of embodiments E138 to E145, comprising 0.1% (R)-4-(5-(4-methoxy-3-propoxyphenyl)pyridin-3-yl)-1,2-oxaborolan-2-ol or a pharmaceutically acceptable salt thereof.

E164. The topical formulation according to any one of embodiments E138 to E145, comprising 0.2% (R)-4-(5-(4-methoxy-3-propoxyphenyl)pyridin-3-yl)-1,2-oxaborolan-2-ol or a pharmaceutically acceptable salt thereof.

E165. The topical formulation according to any one of embodiments E138 to E145, comprising 0.3% (R)-4-(5-(4-methoxy-3-propoxyphenyl)pyridin-3-yl)-1,2-oxaborolan-2-ol or a pharmaceutically acceptable salt thereof.

E166. The topical formulation according to any one of embodiments E138 to E145, comprising 0.4% (R)-4-(5-(4-methoxy-3-propoxyphenyl)pyridin-3-yl)-1,2-oxaborolan-2-ol or a pharmaceutically acceptable salt thereof.

E167. The topical formulation according to any one of embodiments E138 to E145, comprising 0.5% (R)-4-(5-(4-methoxy-3-propoxyphenyl)pyridin-3-yl)-1,2-oxaborolan-2-ol or a pharmaceutically acceptable salt thereof.

E168. The topical formulation according to any one of embodiments E138 to E145, comprising 0.6% (R)-4-(5-(4-methoxy-3-propoxyphenyl)pyridin-3-yl)-1,2-oxaborolan-2-ol or a pharmaceutically acceptable salt thereof.

E169. The topical formulation according to any one of embodiments E138 to E145, comprising 0.7% (R)-4-(5-(4-methoxy-3-propoxyphenyl)pyridin-3-yl)-1,2-oxaborolan-2-ol or a pharmaceutically acceptable salt thereof.

E170. The topical formulation according to any one of embodiments E138 to E145, comprising 0.8% (R)-4-(5-(4-methoxy-3-propoxyphenyl)pyridin-3-yl)-1,2-oxaborolan-2-ol or a pharmaceutically acceptable salt thereof.

E171. The topical formulation according to any one of embodiments E138 to E145, comprising 0.9% (R)-4-(5-(4-methoxy-3-propoxyphenyl)pyridin-3-yl)-1,2-oxaborolan-2-ol or a pharmaceutically acceptable salt thereof.

E172. The topical formulation according to any one of embodiments E138 to E145, comprising 1.0% (R)-4-(5-(4-methoxy-3-propoxyphenyl)pyridin-3-yl)-1,2-oxaborolan-2-ol or a pharmaceutically acceptable salt thereof.

E173. The topical formulation according to any one of embodiments E138 to E145, comprising 1.5% (R)-4-(5-(4-methoxy-3-propoxyphenyl)pyridin-3-yl)-1,2-oxaborolan-2-ol or a pharmaceutically acceptable salt thereof.

E174. The topical formulation according to any one of embodiments E138 to E145, comprising 2.0% (R)-4-(5-(4-methoxy-3-propoxyphenyl)pyridin-3-yl)-1,2-oxaborolan-2-ol or a pharmaceutically acceptable salt thereof.

### DEFINITIONS

The term “PF-07038124” or “active” or “active pharmaceutical agent” or “API” or “drug product,” as used herein, means (R)-4-(5-(4-methoxy-3-propoxyphenyl)pyridin-3-yl)-1,2-oxaborolan-2-ol of structure

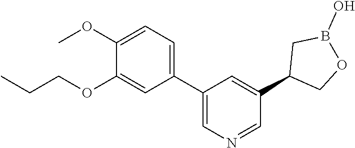 [SMILES: CCCOc1cc(-c2cncc([C@@H]3COB(O)C3)c2)ccc1OC]

and includes pharmaceutically acceptable salts thereof and crystalline or amorphous forms including hydrates, solvates, co-crystals, salts and combinations thereof. Certain forms may be prepared following the experimental procedures disclosed in WO2020/070651 herein incorporated by reference in its entirety.

The term “PF-07086920,” “degradant diol,” or “M1,” as used herein, means 2-(5-(4-methoxy-3-propoxyphenyl)pyridin-3-yl)propane-1,3-diol of structure

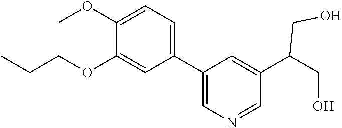 [SMILES: CCCOc1cc(-c2cncc(C(CO)CO)c2)ccc1OC]

which is the main degradant found in the Phase I formulations and also the main metabolite formed in human blood and potentially in other tissues.

The term “high shear bottom mounted dispersion-homogenizer unit” or “dispersion-homogenizer” or “DISHO,” as used herein, is the apparatus that homogenizes the active phase and the wax phase to provide topical formulations of the present invention. The DISHO also ensures recirculation of the topical formulation and can pump the topical formulation from the homogenization tank to the holding tank.

The term “Formulation L,” as used herein, means the three formulations prepared at 270 kilogram scale (total weight) comprising PF-07038124, HG, white petrolatum, mono- and di-glycerides, and paraffin wax disclosed in Table 5 herein. A preferred Formulation L comprises 0.01% PF-07038124, 5% HG, 82.99% white petrolatum, 7% mono- and di-glycerides, 5% paraffin wax, and 0% butylated hydroxytoluene. A topical ointment may be prepared from Formulation L for treating inflammatory disorders in subjects in need of such treatment. Atopic dermatitis and psoriasis are preferred inflammatory disorders for treatment with a topical ointment prepared from Formulation L.

The term “homogenization” or “homogenizes,” as used herein, means a process where the wax phase and the active phase is mechanically mixed to promote uniform dispersion of immiscible emulsion phases and provide the topical formulations of the present invention.

The term “lobe pump,” as used herein, is an external low-shear pump used to transfer topical formulations as an ointment between tanks, such as from the homogenization tank to the jacketed holding tank and from the jacketed holding tank to the filler hopper.

The term “mono- and di-glycerides” as used herein means a mixture of glycerol mono- and di-esters, with minor amounts of tri-esters, of fatty acids from edible oils. It contains not less than 40.0% of monoglycerides. The monoglyceride content is not less than 90.0% and not more than 110.0% of the value indicated in the labeling. It may contain suitable stabilizers. US Pharmacopeia USP43-NF38, page 5890. The formulations of the present invention used Geleol™ supplied by Gettefosse. Geleol™ has specific fatty acid definition such that stearic acid (C18) is 40.0 to 60.0%; and sum of palmitic (C16) and stearic acids is >=90.0%. Fatty acids stabilize functionality of mono- and diglycerides in the formulations of the present invention.

The term “tri-glycerides” as used herein means tri-esters of glycerol with minor amounts of mono- and diesters of glycerol being present. Representative triglycerides that can be used in certain formulations of the present invention include trilaurin (TG12₀12₀12₀), tripalmitin (TG16₀16₀16₀), tristearin (TG18₀18₀18₀), triolein (TG18₁18₁18₁) and trilinolein (TG18₂18₂18₂).

The term “mono-, di- and tri-glycerides” as used herein means a mixture of glycerol mono-, di- and tri-esters of fatty acids from edible oils. Compritol® 888 ATO supplied by Gettefosse is a representative mono-, di- and tri-glyceride and is a mixture of mono-, di- and triesters of behenic acid (C22) with the diester fraction being predominant. More specifically Compritol® comprises a mixture of glycerol monobehenate (12-18% w/w), glycerol dibehenate (45-54% w/w) and glycerol tribehenate (28-32% w/w).

The term “overnight,” as used herein, mean up to 36 hours.

The term “ppm,” as used herein means parts per million. The term “quantum satis,” means sufficient amount to make the total=100%. The term “recirculation,” as used herein, means a bottom-to-top movement of the topical formulation in the homogenization tank via a recirculation loop aided by a pump. DISHO accomplishes recirculation of the topical formulation as long as it is running at a certain minimum rpm. The DISHO is an integrated part of the homogenization vessel and it acts to homogenize and pump the topical formulation.

The term “static,” as used herein, means the formulation was allowed to rest motionless or without mixing, stirring, agitation, or homogenizing.

The term “subject” or “patient” or “individual,” as used interchangeably herein, refers to a human or animal to which the methods of the present invention can be applied. In certain preferred embodiments, the subject is a mammal or human, more preferably a human.

The term “w/w,” as used herein, means weight for weight or weight by weight.

The term “essentially free of butylated hydroxytoluene” or “essentially free of BHT,” means that butylated hydroxytoluene is not deliberately added to improve the properties of the formulation (e.g. chemical stability) and, if present at all, does not exceed trace amounts and preferably is less than 25 ppm.

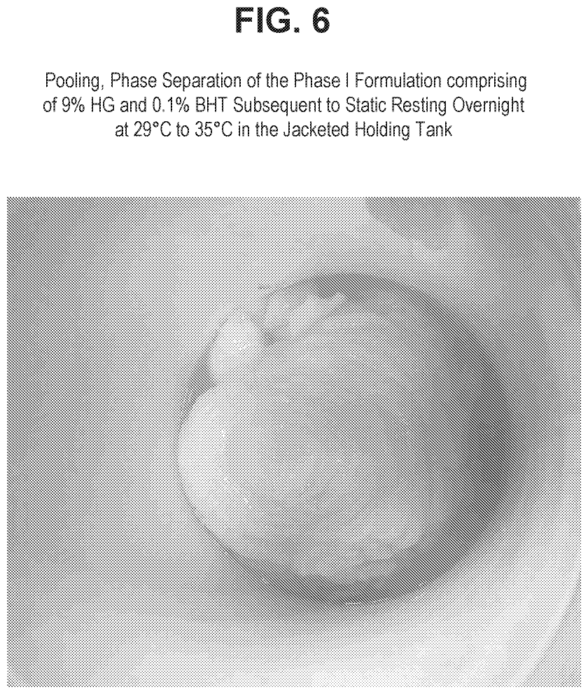

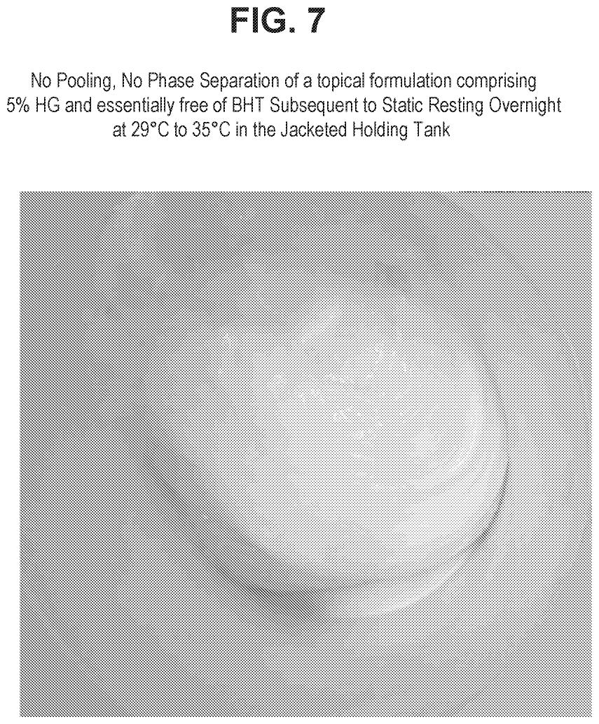

The Phase I formulations (Table 1) were manufactured at 180 kg scale using a 300 Liter Fryma Koruma scrape surface mixer. The manufactured formulations were subsequently transferred into a holding tank and held for a day prior to filling into tubes. The transfer process was assisted by using a high shear bottom mounted dispersion-homogenizer unit (DISHO) that is part of the Fryma Koruma vessel. On the day of tube filling, a pool of about 100 mLs of liquid was observed near the bottom of the holding tank (FIG. 6). A sample of this liquid was collected, analyzed, and determined to contain mainly hexylene glycol, suggesting that hexylene glycol separated from the formulation. This observation of liquid pooling, or phase separation during manufacturing, implies that the Phase 1 formulations (Table 1) lack robustness in its ability to be processed while remaining resistant to liquid pooling or phase separation.

TABLE 1
Phase I Formulations

| Component | Percentage (%) | | |
|---|---|---|---|
| PF-07038124 | 0.01 | 0.03 | 0.06 |
| Hexylene Glycol | 9 | 9 | 9 |
| White Petrolatum* | 78.89 | 78.87 | 78.84 |
| Mono- and Di-glycerides | 7 | 7 | 7 |
| Paraffin Wax | 5 | 5 | 5 |
| Butylated Hydroxytoluene | 0.1 | 0.1 | 0.1 |
| TOTAL | 100 | 100 | 100 |

*White petrolatum was added quantum satis (qs).

Reducing the amount of hexylene glycol (HG) to 5% and 2% (replaced with white petrolatum) in the Phase I formulations as a possible remedy to the manufacturing issues unexpectedly increased levels of degradant diol PF-07086920 (Table 2) suggesting that reducing HG lessened the effect of the antioxidant butylated hydroxytoluene (BHT). This discovery was surprising and contrary to expectation because the amount of antioxidant BHT and PF-07038124 and their ratio were not changed in the 5% HG and 2% HG formulations and therefore the levels of degradant diol (PF-07086920) in the 5% HG and 2% HG formulations were expected to be similar to the 9% HG formulation.

TABLE 2
PF-07086920 Degradant Levels After Reducing
HG in the Phase I Formulations

| Phase I Components | PF-07086920 Levels |
|---|---|
| 0.01% API, 9% HG, 0.1% BHT | 0.25% |
| 0.01% API, 5% HG, 0.1% BHT | 0.62% |
| 0.01% API, 2% HG, 0.1% BHT | 0.46% |
| 0.03% API, 9% HG, 0.1% BHT | 0.24% |
| 0.03% API, 5% HG, 0.1% BHT | 0.44% |
| 0.03% API, 2% HG, 0.1% BHT | 0.34% |

Further surprising was the discovery that eliminating BHT from the Phase I formulations reduced levels of degradant diol (PF-07086920) in two formulations, 0.01% API, 5% HG, 0% BHT and 0.01% API, 2% HG, 0% BHT, compared with similar formulations containing 0.1% BHT (Table 3). The results shown in Table 3 were surprising because the degradant diol results from an oxidative deboronation transformation that an antioxidant, such as BHT, would be expected to inhibit. The addition of an antioxidant in formulations is a well-practiced approach to counter or alleviate the effect of oxidative degradation of a compound. BHT was added to the Phase I formulations with the specific intention of reducing/eliminating oxidative degradation. The discovery that removal of BHT from the Phase I formulations reduced oxidative degradation of PF-07038124 was contrary to known practice in the formulation field.

TABLE 3
PF-07086920 Levels After Removing
BHT in the Phase I Formulations

| Phase I Components | PF-07086920 Levels |
|---|---|
| 0.01% API, 9% HG, 0% BHT* | 0.30% |
| 0.01% API, 5% HG, 0% BHT* | 0.37% |
| 0.01% API, 2% HG, 0% BHT* | 0.35% |

*A trace amount of BHT in the white petrolatum obtained from the supplier was determined by Applicant to be approximately 20 ppm

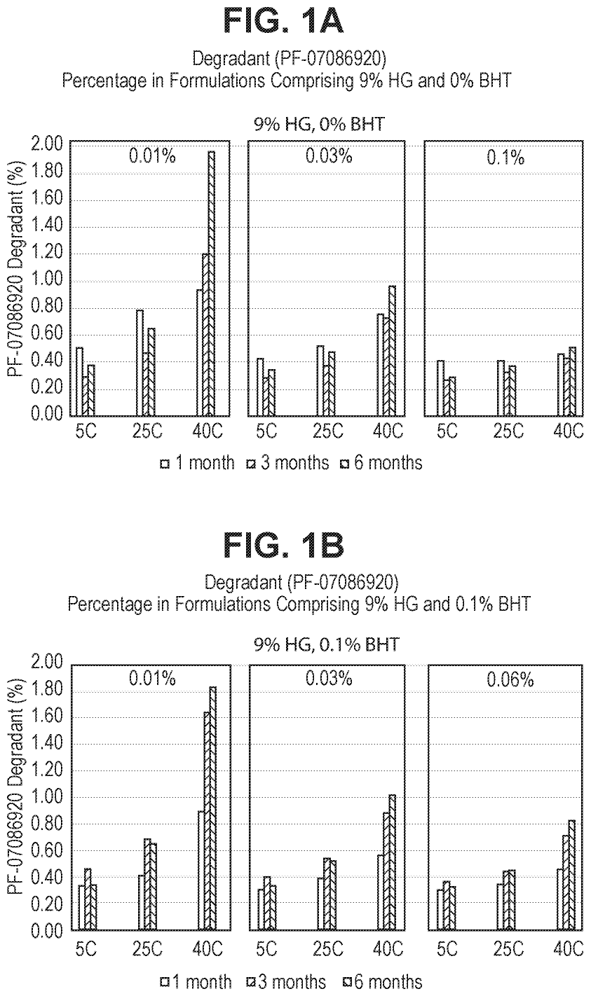

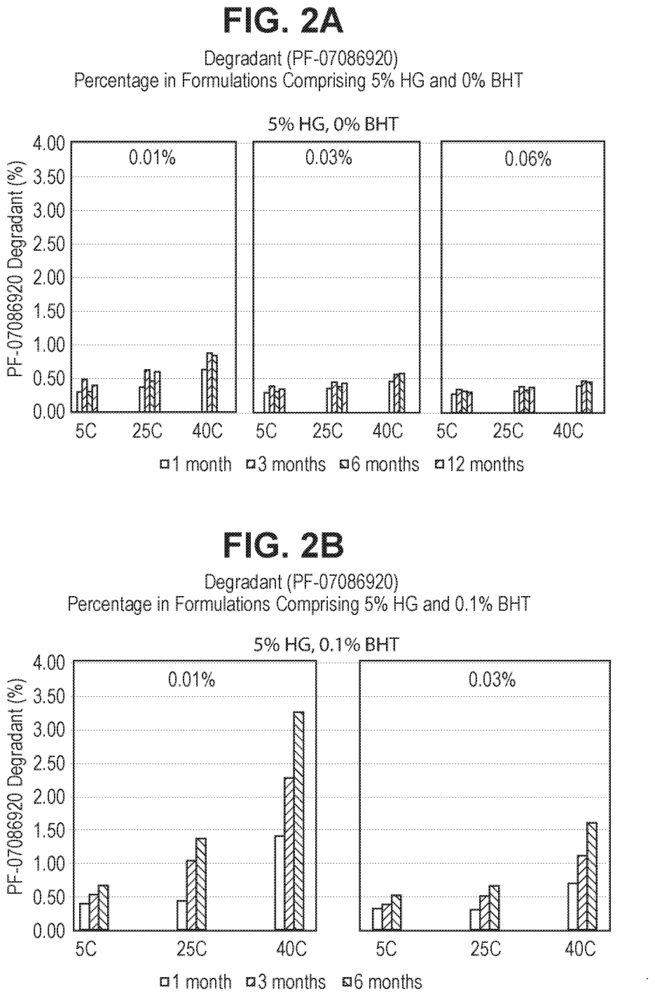

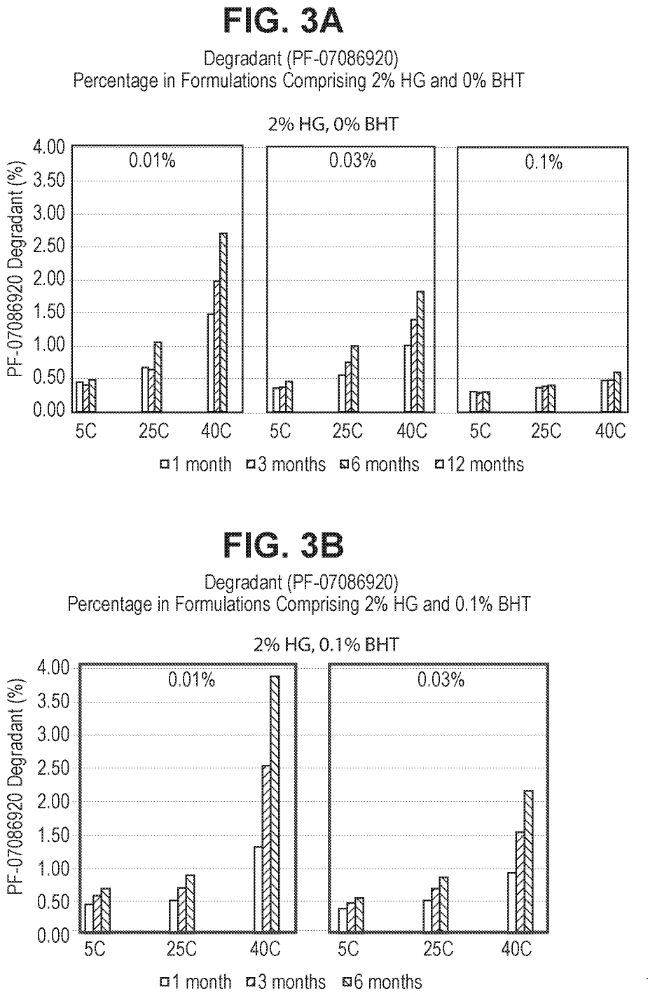

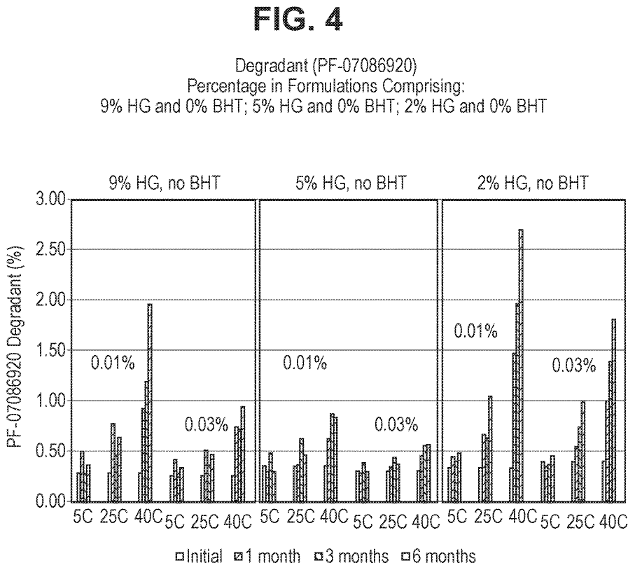

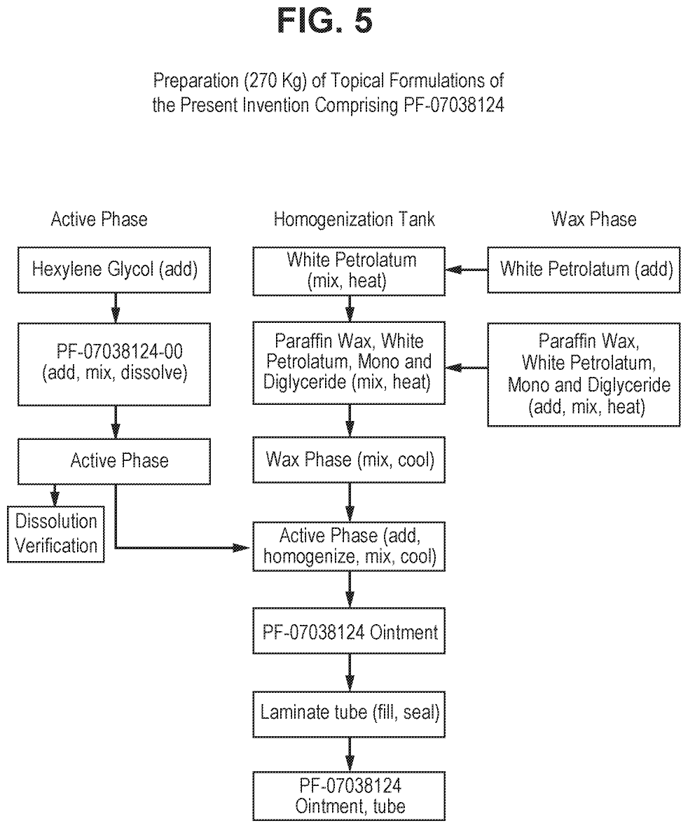

The unexpected results discovered in Tables 2 and 3 warranted further evaluation to determine whether formulations with reduced HG in combination with removal of BHT would improve the physical and chemical issues associated with the Phase I formulations. Comparative data was generated for formulations comprising 9%, 5%, and 2% HG with and without BHT and the amount of degradant diol was measured at certain temperatures and time periods as described in FIGS. 1-4. This data showed that the removal of BHT from the formulations containing 9% and 2% HG, FIGS. 1 and 3 respectively, had some benefit in reducing generation of the degradant diol PF-07086920. Unexpectedly, the formulations with 5% HG and 0% BHT were most effective at reducing the degradant diol PF-07086920 as 20 shown in FIG. 2. Based on the improved chemical stability for formulations with 5% HG and 0% BHT, new formulations were proposed for the Phase II clinical studies shown in Table 4.

TABLE 4
Proposed Phase II Formulations

| Component | Percentage (%) | | | | | |
|---|---|---|---|---|---|---|
| PF-07038124 | 0.005 | 0.01 | 0.02 | 0.03 | 0.06 | 0.1 |
| Hexylene | 5 | 5 | 5 | 5 | 5 | 5 |
| Glycol | | | | | | |
| White | 82.995 | 82.99 | 82.98 | 82.97 | 82.94 | 82.90 |
| Petrolatum | | | | | | |
| Mono- and Di- | 7 | 7 | 7 | 7 | 7 | 7 |
| glycerides | | | | | | |
| Paraffin Wax | 5 | 5 | 5 | 5 | 5 | 5 |
| TOTAL | 100 | 100 | 100 | 100 | 100 | 100 |

### Manufacture

The proposed Phase II formulations (Table 5), designated as Formula L, were manufactured at 270 kg scale using the same equipment used to prepare the Phase I formulations. No liquid pooling was observed near the bottom of the jacketed holding tank after Formulation L remained in the tank overnight at 29° C. to 35° C. in static fashion (no mixing, no stirring). The transfer process from the homogenization tank to the jacketed holding tank was assisted by using the bottom mounted high shear dispersion-homogenizer unit (DISHO) or using an external low shear lobe pump. Therefore, Formulation L was found to be more robust in its ability to be processed while remaining resistant to liquid pooling or phase separation, regardless of whether a high shear DISHO or a low shear lobe pump was used to empty the homogenization tank.

More specifically, PF-07038124 was pre-weighed and dissolved in hexylene glycol, visually verified, with mixing over about 2 to 3.5 hours in an appropriately sized vessel to provide the active phase. White petrolatum was added to a separate vessel and heated to 70° C. to 80° C. followed by addition of paraffin wax and mono- and di-glycerides with mixing until all the excipients melted/dissolved as determined by visual verification to provide the wax phase. The wax phase was transferred to the homogenization tank and heated to 70° C. to 80° C. with recirculation not less than 10 minutes. The homogenization tank was cooled to 40° C. to 46° C. with mixing and recirculation. The active phase was then added to the homogenization tank and the wax and active phases were homogenized not less than 60 minutes with DISHO at 1600 rpm to provide Formulation L. The DISHO rpm was adjusted as needed to maintain the temperature at 40° C. to 46° C. Subsequent to homogenization, the tank was cooled 29° C. to 35° C. and the DISHO rpm reduced to 1000. The DISHO rpm was adjusted to maintain the temperature at 29° C. to 35° C. and to ensure the speed remained above the minimum speed to allow for appropriate recirculation. Formulation L was transferred to a jacketed holding tank at at 29° C. to 35° C. and allowed to rest overnight in the holding tank without further mixing or stirring. The jacketed holding tank was then connected via hose to a filling hopper set at 29° C. to 35° C. and Formulation L was pumped into tubes and the tubes sealed and passed over an automatic weight checker to provide Formulation L in dispensable tubes.

TABLE 5
Manufactured Formulations: Placebo and Formulation L

| Component | Percentage (%) | | | | Amounts (kgs) | | | |
|---|---|---|---|---|---|---|---|---|
| PF-07038124 | 0.0 | 0.005 | 0.01 | 0.02 | 0.0 | 0.0135 | 0.027 | 0.054 |
| Hexylene Glycol | 5 | 5 | 5 | 5 | 13.5 | 13.5 | 13.5 | 13.5 |
| White Petrolatum | 83 | 82.995 | 82.99 | 82.98 | 224.10 | 224.0865 | 224.073 | 224.046 |
| Mono- and Di-glycerides | 7 | 7 | 7 | 7 | 18.9 | 18.9 | 18.9 | 18.9 |
| Paraffin Wax | 5 | 5 | 5 | 5 | 13.5 | 13.5 | 13.5 | 13.5 |
| TOTAL | 100 | 100 | 100 | 100 | 270 | 270 | 270 | 270 |

| | | | |
|---|---|---|---|
| Placebo | Formula L | Placebo | Formula L |

Formulation L demonstrated enhanced chemical and physical stability that: resolved the liquid pooling issue; eliminated phase separation; enabled the homogenization tank to be emptied with more pump options; and reduced degradant diol (PF-07086920) levels.

### Metabolites

An assessment of the metabolism of non-radiolabeled PF-07038124 was 5 conducted in vitro using mouse, rat, dog, monkey and human hepatocytes. Moderate to extensive metabolism of PF-07038124 was observed in all species' hepatocytes. The primary routes of metabolism were hydrolysis/oxidation of the oxaborole ring to yield the diol (PF-07086920), glucuronidation of the pyridyl nitrogen, hydroxylation on the propyl group of the phenoxypropyl moiety, and demethylation of the methoxy group (shown below). All metabolites detected in mouse, rat, dog, and monkey hepatocytes were also observed in human hepatocytes. These in vitro assessments showed that the boronic acid moiety was chemically unstable when exposed to a variety of mammalian hepatocytes.

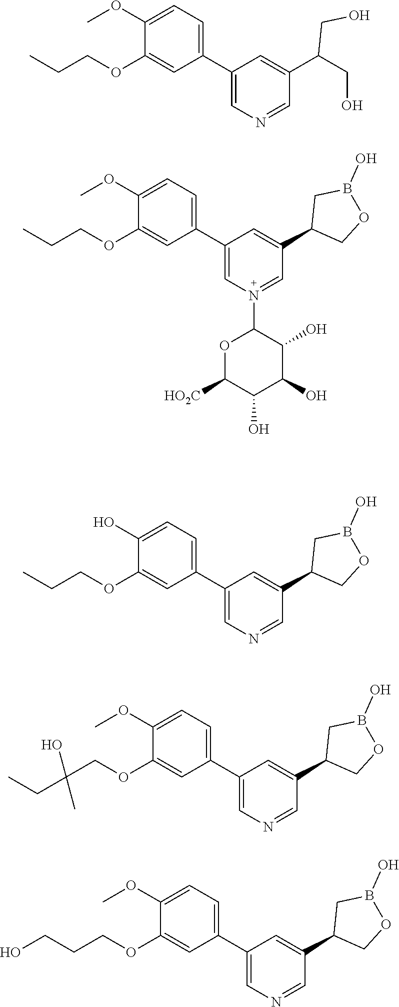

One of the major metabolic pathways in humans was formation of the oxaborole ring opened molecule, PF-07086920. PF-07038124 was found to be chemically unstable due to oxidation of the oxaborole ring to give the diol degradant/metabolite (PF-07086920) in human and rat blood. Following incubation for 1 hour at 1 μM in human and rat blood, the concentration of PF-07038124 declined whereas the concentration of PF-07086920 increased in both human and rat blood as determined by liquid chromatography-tandem mass spectrometry. In addition to being a metabolite, PF-07086920 was also determined to be a degradant in the formulations comprising PF-07038124.

1. A topical formulation comprising 0.0025% to 2% (R)-4-(5-(4-methoxy-3-propoxyphenyl)pyridin-3-yl)-1,2-oxaborolan-2-ol or a pharmaceutically acceptable salt thereof, 2% to 9% hexylene glycol, quantum satis (qs) white petrolatum, 5% to 9% mono- and di-glycerides, and 3% to 7% paraffin wax, wherein the topical formulation is essentially free of butylated hydroxytoluene and wherein the topical formulation has 3.0% w/w or less degradant diol at 0 to 6 months at 5° C. to 40° C.

2. The topical formulation according to claim 1, comprising 5% hexylene glycol, 7% mono- and diglycerides, and 5% paraffin wax.

3. The topical formulation according to claim 1, comprising 0.005% to 1.0% (R)-4-(5-(4-methoxy-3-propoxyphenyl)pyridin-3-yl)-1,2-oxaborolan-2-ol or a pharmaceutically acceptable salt thereof.

4. The topical formulation according to claim 2, comprising 0.005% to 0.5% (R)-4-(5-(4-methoxy-3-propoxyphenyl)pyridin-3-yl)-1,2-oxaborolan-2-ol or a pharmaceutically acceptable salt thereof.

5. The topical formulation according to claim 2, comprising 0.01% (R)-4-(5-(4-methoxy-3-propoxyphenyl)pyridin-3-yl)-1,2-oxaborolan-2-ol or a pharmaceutically acceptable salt thereof.

6. A topical formulation comprising 0.01% (R)-4-(5-(4-methoxy-3-propoxyphenyl)pyridin-3-yl)-1,2-oxaborolan-2-ol or a pharmaceutically acceptable salt thereof, 5% hexylene glycol, quantum satis white petrolatum, 7% mono- and di-glycerides, and 5 paraffin wax, wherein the topical formulation is essentially free of butylated hydroxytoluene and wherein the topical formulation has 1.0% w/w or less degradant diol at 0 to 6 months at 5° C. to 40° C.

7. A topical formulation comprising 0.01% (R)-4-(5-(4-methoxy-3-propoxyphenyl)pyridin-3-yl)-1,2-oxaborolan-2-ol or a pharmaceutically acceptable salt thereof, 5% hexylene glycol, quantum satis white petrolatum, 7% mono- and di-glycerides, and 5% paraffin wax, wherein the topical formulation is essentially free of butylated hydroxytoluene and wherein the topical formulation has 1.0% w/w or less degradant diol at 0 to 12 months at 5° C. to 25° C.

8. A method for treating a disease or condition selected from the group consisting of psoriasis, plaque psoriasis, pustular psoriasis, nail psoriasis, flexural psoriasis, guttate psoriasis, psoriatic arthritis, erythrodermic psoriasis, and inverse psoriasis in comprising administering to the subject a subject in need of such treatment a therapeutically effective amount of the topical formulation according to claim 1.

9. The method according to claim 8, wherein the disease or condition is psoriasis.

10. The method according to claim 8, wherein the disease or condition is plaque psoriasis.

11. A method for treating a disease or condition selected from the group consisting of eczema, atopic dermatitis, hand dermatitis, contact dermatitis, allergic contact dermatitis, irritant contact dermatitis, neurodermatitis, perioral dermatitis, stasis dermatitis, dyshidrotic eczema, xerotic dermatitis, nummalar dermatitis, seborrheic dermatitis, eyelid dermatitis, diaper dermatitis, dermatomyositis, lichen planus, lichen sclerosis, alopecia areata, vitiligo, rosacea, epidermolysis bullosa, keratosis pilaris, pityriasis alba, pemphigus, vulvovaginitis, acne, chronic spontaneous urticaria, chronic idiopathic urticaria, chronic physical urticaria, vogt-koyanagi-harada disease, sutton nevus/nevi, post inflammatory hypopigmentation, senile leukoderma, chemical/drug-induced leukoderma, cutaneous lupus erythematosus, discoid lupus, palmoplantar pustulosis, pemphigoid, sweet's syndrome, and hidradenitis suppurativa in a subject comprising administering to the subject in need of such treatment a therapeutically effective amount of the topical formulation according to claim 1.

12. The method according to claim 11, wherein the disease or condition is atopic dermatitis.

13. The method according to claim 11, wherein the disease or condition is eczema.
